(19) United States
(12) Patent Application Publication
(10) Pub. No.: US 2004/0176752 A1
(43) Pub. Date: Sep. 9, 2004

(54) System and methods for laser treatment of ocular tissue

(76) Inventors: Robert R. Alfano, Bronx, NY (US); Tapan K. Gayen, Brooklyn, NY (US); Steven A. McCormick, New York, NY (US); Richard B. Rosen, New York, NY (US); Alvin Katz, Bronx, NY (US); Howard E. Savage, Ossining, NY (US)

Correspondence Address: BAKER & BOTTS, 30 ROCKEFELLER PLAZA, NEW YORK, NY, 10112

(21) Appl. No.: 10/383,124

(22) Filed: Mar. 6, 2003

(51) Int. Cl. A61B 18/20

(52) U.S. Cl. 606/4; 606/8

(57) ABSTRACT

A system and method for exposing ocular tissue to a beam of electromagnetic radiation tuned to an NIR wavelength between about 1380 nm and about 1600 nm in the water absorption band. The laser energy is absorbed by the water, which in turn, transfers the energy to collagen molecules. The helical collagen molecules partially denature and then re-nature to form a weld across the region of the tissues to be joined. In a preferred embodiment, the electromagnetic radiation originates from a laser. The lasers may be Cr⁴⁺ doped crystals, Erbium fibers, and alloys of semiconductor laser diodes.

### BACKGROUND OF THE INVENTION

1. Field of the Invention

This invention relates to systems and methods for laser surgical techniques, and more particularly to the use of lasers in the near infrared (NIR) range for treatment of ocular tissue.

2. Background

Conventional approaches to surgically joining ocular tissue are well known and include the use of sutures, staples, clips, and adhesives. For example, sutures and adhesives have been used to seal incisions and laserations in the cornea. Repair of a retinal detachment (RD) may be performed by such procedures as the scleral buckle, the vitrectomy, and pneumatic retinopexy. In the scleral buckle procedure, which is used for uncomplicated rhegmatogenous RDs, the retinal breaks are located, treated with a cryoprobe and then supported with a scleral buckle. The buckle itself is a piece of silicone sponge or solid silicone that is sewn onto the outer wall of the eyeball and positioned to cause an indentation or buckle inside the eye so that it pushes in on and closes the retinal break. Potential adverse effects of conventional suture techniques and the scleral buckle include inflammation, granulation and excessive collagen accumulation around the sutures. Moreover, the silicone sponge used in the scleral buckle procedure represents a foreign body that must remain within the body.

The vitrectomy procedure, typically referred to as a trans pars plana vitrectomy or TPPV, is often used for tractional retinal detachments, but can also be used for rhegmatogenous RDs if they are associated with vitreous traction or hemorrhage. According to this procedure, incisions are made in the wall of the eye to allow instruments to be used in the vitreous cavity. First, the vitreous is removed using a vitreous cutter. Next, a variety of instruments and techniques are used to reattach the retina. For example, tractional bands are removed with scissors and forceps and then silicone oil is placed in the vitreous cavity to push and hold the detached retina against the retinal pigment epithelial layer. A disadvantage of this procedure is the requirement of the patient to maintain a specific head position after surgery in order to keep the retina attached.

A pneumatic, retinopexy is typically used to repair uncomplicated rhegmatogenous RDs that have a single break in the superior portion of the retina. Usually performed under local anesthesia, this procedure involves injection of a small (0.4-mL), expandable fluorocarbon gas bubble trans sclerally (through the pars plana) into the vitreous cavity. The patient is positioned so that the gas bubble (which rises) closes the retinal break, thereby stopping further vitreal fluid from passing through the tear or hole into the detachment space. If the placement of the gas bubble can be accomplished, the subretinal fluid will usually resolve within two days. One disadvantage of this procedure is that the patient is required to keep a precise head position 7-10 days after the procedure. This procedure typically has a lower success rate when compared to the scleral buckle procedure.

Additional methods for developing chorioretinal adhesion (retinopexy) include diathermy and cryotherapy, which aim to create a chorioretinal scar around the retinal break that will subsequently fuse the two layers as the tissue heals. The formation of scar tissue requires that the inner and outer segment layers of the rods and cones of the retina, and retinal pigment epithelium (RPE) layer be in contact with each other. With diathermy, an alternating electrical current of 13.56 MHz is generated and passed through the retinal tissue as a heat source. The heat produced coagulates the tissue, thereby producing an adhesion to the RPE. With cryotherapy, a cryoprobe is applied to the sclera until whitening is observed under direct ophthalmoscopic visualization. The freezing of the RPE is sufficient to form chorioretinal bonding. Diathermy has the problem of causing occular occlusion of major blood vessels, scleral shrinkage and tissue necrosis. Cryotherapy has the disavantage of causing wider disruption of the blood retinal barrier and greater mobilization of the RPE cells and postoperative cystoid macular edema.

Laser fusion of tissues provides a substantial advance over conventional procedures, such as those listed above, and is accomplished by directing a low energy laser beam of the appropriate wavelength at the opposed edges of the tissue to be joined. Ophthalmologists were among the first to use laser technology for the fusion of human tissues, specifically, the treatment of retinal detachments. The term “photocoagulation” refers to a procedure of joining of tissue, in which the thermal effects of the laser may cause denaturation of molecules within the tissue, and that these effects can be seen when using histology. For example, in the reattachment of a detached retina, photocoagulation is acceptable and desirable because the reattachment is due to scar tissue that forms over time between the choroid and the retina. With photocoagulation, laser light is directed through the sclera or the pupil to the retina, RPE and choroid. The heat produced at this level causes the coagulation and subsequent bonding of the tissues being joined, such as, e.g., the retina to the RPE and choroid.

Laser tissue “welding” typically causes minimal disruption of tissue. Laser welding is especially useful when treating tissues such as the cornea, in which minimal disruption of the tissue is occurs so that vision is preserved. Opacities or irregularities in the tissue may result in a decrease in visual acuity.

Laser photocoagulation and welding have a number of advantages over conventional methods. For example, these techniques typically cause less foreign body reaction, more rapid healing, less constriction and more rapid surgical time, and provide immediate wound strength, fluid tight closure, reduced probability of infection and improved cosmetic results. Laser-fused wounds typically have reduced inflammatory response, near normal collagen content, and minimal residual disorientation and breaks in the elastic fibers. Laser fusion of eye tissues also provides the advantages of instantaneous adherence of the separated tissues and less tissue disruption.

For example, Meyer-Schwickerath used light coagulation in closing holes over retinal buckles in cases in which the hole was not in contact with the buckled wall of the eye (as described in Meyer-Schwickerath, G., “Treatment of Eales Disease and Diabetic Retinopathy with Photocoagulation,” Trans Opthalmol Soc UK, 84:67-76, 1964.) One explanation given for the process of photocoagulation of the retina is that light is absorbed by the retina pigment epithelium (RPE) and that destruction of these cells occurs. The photoreceptors and the outer portion of the retina are also damaged by the absorption of the light and by the heat that is produced. The result of the photocoagulation is the development of a chorioretinal scar, similar to that seen with diathermy, as described above.

More recently, laser fusion of scleral and corneal tissues has been described. Pini, for example, used a low power diode laser at a wavelength of 805 nm to perform a human corneal weld (as described Pini, R., Menabuoni, L., and Starnoti, L., “First Application of Laser Welding in Clinical Transplantation of the Cornea, Proceedings SPIE, 4233: 266-271, 2001). This procedure was performed with the use a chromophore that was applied locally to the wound to produce local absorption and controlled release of heat. A disadvantage of this technique is the need to use extrinsic dyes or solders.

Burstein describes a fusion of corneal tissue from porcine cadaver eyes using a fundamental hydrogen fluoride (HF) wavelength of 2.5588 μm at 30 mW and a HF overtone wavelength of 1.3404 μm at 320 mW produced from a hydrogen fluoride chemical laser (as discussed in Burstein, N. L., Williams, J. M., Sr., Nowicki, M. J., Johnson, D. E., Jeffers, W. Q., “Corneal Welding Using Hydrogen Fluoride Lasers,” Arch Opthamology, 110:12-13, 1992.) Unsuccessful attempts of laser corneal welding were documented by Keates et al. (Keates, R. H., Levy, S. N., Fired, S., and Morris, J. R., “Carbon Dioxide Laser Use in Wound Sealing and Epikeratophakia,” J Cataract Refract Surg, 13:290-295, 1987) and Gailitis et al. (Gailitis, R. P., Thompson, K. P., Ren, Q. S., Morris, J., and Waring, G. O., “3rd Laser Welding of Epikeratoplasty Lenticules to the Cornea,” Refract Corneal Surg., 6:430-436, 1990). Keates et al. used a carbon dioxide (CO₂) laser (10.6 μm) to attempt to weld human scleral and corneal eye bank tissue and albino rabbit ocular tissue, but achieved no fusion of the tissues. Gailitis et al. attempted to use a continuous wave CO₂ laser to weld strips of synthetic collagen together and synthetic collagen epikeratoplasty lenticules to the cornea. Only temporary welding was achieved when a 30% bovine serum albumin was used as a solder.

Barak et al. used a temperature-controlled CO₂ laser in the mid to far infrared (wavelength of 10.6 μm) to weld corneal and corneoscleral wounds in bovine eyes in vitro and rabbit eyes in vivo (as discussed in Barak, A., Eyal, O., Rosner, M., Belotserkousky, E., Soloman, A., Belkin, M., and Katzir, A., “Temperature Controlled CO₂ Laser Tissue Welding of Ocular Tissues,” Surv Ophthamal, 42 Suppl 1:S77-81, 1997). Strassmann used a temperature controlled CO₂ laser at a wavelength of 10.6 μm to weld in vitro bovine cornea and in vivo rabbit cornea (as discussed in Strassman, E., Loya, N., Gaton, D. D., Ravid, A., Kariv, N., Weinberger, D., and Katzir, A., “Laser Soldering of the Cornea in a Rabbit Model Using a Controlled-Temperature CO₂ Laser System,” Proceedings SPIE, 4224:253-265, 2001).

The current methods of joining ocular tissues with lasers in the above listed wavelengths have the disadvantage in that the wavelengths that are used do not fall within the water absorption range. For this reason, these lasers are not believed to heat the tissue efficiently and therefore may not allow for a strong weld and also a full thickness weld. Accordingly, there is a need in the art for a technique of re-annealing ocular tissue that overcomes the limitations of the conventional techniques. In particular, a technique is needed which provides strong welds and which does not require the use of extrinsic dyes, chromophores, or solders.

### SUMMARY OF THE INVENTION

An object of the present invention is to provide apparatus and techniques for treating ocular tissue by use of a laser operating in the near infrared wavelength.

Another object of the present invention is to provide apparatus and techniques for treating ocular tissue by use of a laser operating in the water absorption band.

A further object of the present invention is to form a full thickness tissue bond in real-time.

A still further object of the present invention is to weld ocular tissue without use of extrinsic protein solders or dyes.

Another object of the present invention is to provide apparatus and techniques for reattaching splits in the retina including intralayer splits in the inner and outer segments of the rods and cones, interlayer splits between the inner and outer segments of the rods and cones layer and the RPE, and interlayer splits between the choroid layer and the sclera.

A further object of the present invention is to provide apparatus and techniques for reattaching splits in the sclera, including incisional splits in the scleral tissue.

A still further object of the present invention is to provide apparatus and techniques for reattaching splits in the cornea, including splits in the cornea formed during operations or corneal button preparations.

These and other objects of the invention, which will become apparent with reference to the disclosure herein, are accomplished by a system and method for exposing ocular tissue to a beam of electromagnetic radiation tuned to an NIR wavelength between about 1380 nm and about 1600 nm in the water absorption band. In a preferred embodiment, the electromagnetic radiation originates from a laser. The lasers may be Cr⁴⁺ doped crystals, Erbium fiber lasers, and alloys of semiconductor laser diodes that emit tunable radiation in the 1380 to 1600 nm range.

In an exemplary embodiment, the step of exposing ocular tissue may comprise exposing RPE tissue to the electromagnetic radiation. In another exemplary embodiment, the step of exposing ocular tissue may comprise exposing a choroid layer and scleral tissue having an interlayer split therebetween to the electromagnetic radiation. According to yet another exemplary embodiment, the step of exposing ocular tissue may comprise exposing scleral tissue to the electromagnetic radiation.

The method may further comprise scanning an excitation area and periodically blocking the excitation area from the electromagnetic radiation. Cool down periods, during the weld, when the laser is shuttered, prevents overheating of the ocular tissue and allows collagen molecules to renature. The step of exposing ocular tissue may comprise exposing tissue to a scanning laser beam, and spatial offsetting the scanning laser beam from the region in which sections of ocular tissue are to be joined. This spatial offset of the scanning laser beam reduces thermally induced buckling of tissue.

In accordance with the invention, the objects as described above have been met, and the need in the art for repairing ocular tissue with the use of a laser in the NIR band without the use of extrinsic dye or solders, has been satisfied.

### BRIEF DESCRIPTION OF THE DRAWINGS

Further objects, features and advantages of the invention will become apparent from the following detailed description taken in conjunction with the accompanying figures showing illustrative embodiments of the invention, in which:

FIG. 1 is a graph indicating the absorption band of water.

FIG. 2 is block diagram of the apparatus in accordance with the present invention.

FIG. 3 is a sectional view of tissue treated by the method in accordance with the present invention.

FIG. 4 is a sectional view of tissue treated by the method in accordance with the present invention.

FIG. 5 is a sectional view of tissue treated by the method in accordance with the present invention.

FIG. 6 is a sectional view of tissue treated by the method in accordance with the present invention.

FIG. 7 is a sectional view of tissue treated by the method in accordance with the present invention.

Throughout the figures, the same reference numerals and characters, unless otherwise stated, are used to denote like features, elements, components or portions of the illustrated embodiments. Moreover, while the subject invention will now be described in detail with reference to the figures, it is done so in connection with the illustrative embodiments. It is intended that changes and modifications can be made to the described embodiments without departing from the true scope and spirit of the subject invention as defined by the appended claims.

### DETAILED DESCRIPTION OF EXEMPLARY EMBODIMENTS

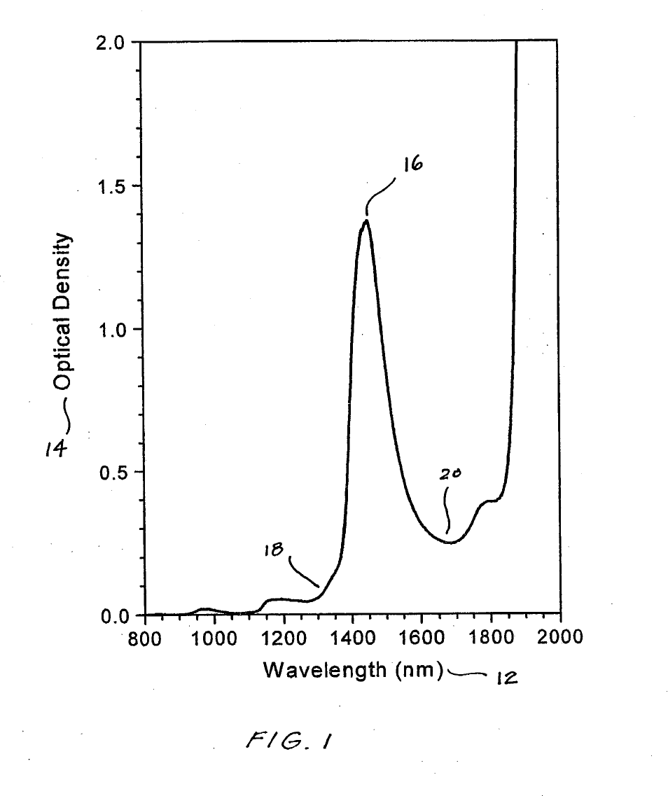

In accordance with the invention, a source of NIR electromagnetic radiation is tuned to a wavelength in the 1380 to 1600 nm range, and output power is coupled into an optical fiber and used to repair ocular tissues. The emission wavelength of the laser in the NIR range corresponds to an absorption band of water. The absorption spectrum of water in the NIR is seen in FIG. 1. The emission wavelength is represented in axis 12 and the optical density is represented in axis 14. Optical Density (OD) is defined as the negative base-ten logarithm of the transmittance, i.e. OD=log₁₀ (T). The strong water absorption band peak (as indicated by arrow 16 if FIG. 1) occurs at 1450 nm and covers above-stated range of 1380 nm (as indicated by arrow 18) to 1600 nm (as indicated by arrow 20) and this range of emission has been found appropriate for welding tissues because it allows uniform heating of tissues with a thickness up to 1.5 mm. This absorption band is due to overtones of the v1, v2, and v3 vibrations bands of H₂O. The fundamental vibrational bands are located above 2000 nm seen in FIG. 1.

The molecules that make up the ocular tissues to be joined and the effects of light on these molecules are explained herein. The retina contains a number of molecules that have the capacity to react with and absorb light and include melanin, hemoglobin, collagen, xanthophylls, lipofuscin, photopigments and water.

The molecular constituents of the inner and outer segments of the rod and cone layer of the retina will now be described. The outer segments of the rod and cone layer of the retina project towards the RPE layer, which is the location in which a split in the retina may result in retinal detachment. The inter photoreceptor matrix (IPM) surrounds the elongated rod and cone photoreceptor inner and outer segments. The IPM has several important functions including retinal adhesion, photoreceptor alignment, growth factor presentation, retinoid transport, and photoreceptor outer segment recognition for phagocytosis. The IPM may be readily isolated due to its hydrophilic nature. For example, when placed in distilled water, a retinal tissue swells, and the IPM layer separates from the rest of the outer retina. The isolated IPM has been used to document different lectin binding domains. Using chondroitinase digestion, Western Blotting, polyclonal antibodies and lectin probes, two prominent glycoproteins were identified in the IPM: SPACR, (SialoglycoProtein Associated with Cones and Rods, 150 kD molecular weight) and SPACRCAN, (SialoglycoProtein Associated with Cones and Rods Chondroitin Sulfate, 230 kD molecular weight). Hyaluronan was also identified in the IPM matrix, and is thought to be the primary scaffold unit of the IPM. Hyaluronan may be the most important aspect of retinal adhesion. The cells that border the IPM have the capacity to bind hyaluronan through their membrane receptors. These cells include Muller cells, photoreceptor cells and pigment epithelial cells. CD44, a membrane receptor, is found in the apical microvillae of Muller cells, and has the ability to bind hyaluronan. The RPE layer bordering the other side of the IPM binds to the same hyaluronan scaffold through its membrane receptor RHAMM. Neuroglycan C is a transmembrane chondroitin sulfate proteoglycan that is associated with the plasma membrane of the photoreceptor cells. It contains a RHAMM-like hyaluronan-binding motif that also adds to retinal adhesion. Because these receptors bind to the hyaluronan, the retina remains bound to the RPE. Another property of the hyaluronan IPM scaffold is that it is highly hydrophilic. According to this structure, water would be concentrated around the IPM. While not bound by any particular theory of operation, it is believed that NIR light applied according to the present invention, successfully tuned to the H₂O absorption band and and focused on this area, provides a selective welding of these tissues. These IPM molecules would then play a major role in the welding/photocoagulation of a detached retina.

The molecular constituents of the RPE layer will now be described. As discussed above, the RPE layer lies next to the inner and outer segments of the rod and cone layer. The RPE has a number of functions that relate to the retina. First, the RPE forms the outer blood-ocular barrier between the choriocapillaris and the neurosensory retina. Second, the RPE is responsible for the phagocytosis of rod and cone outer segments. Third, the RPE is involved in vitamin A metabolism. Fourth, the apical portion of the RPE lies next to the photoreceptor layer. Each RPE cell has villous processes that interdigitate with the outer segments of the photoreceptor cells. This weak link between the two layers is the basis for continuing retina attachment to the RPE. Molecules that are found in the RPE cells include melanin pigment, and membrane and disk related molecules shed by the rods and cones, due to the phagocytic functions of the RPE. The RPE has a high capacity for water transport; consequently, fluid does not usually accumulate in the subretinal space. This dehydration of the subretinal space serves to stabilize bonding of the inter photoreceptor matrix, which bridges the RPE and photoreceptors layers. Lipofuscin, or aging pigment, is also found in this layer. Melanin, because of its ability to absorb and scatter light, would play a significant role in the absorption of light and the fusion of tissues. When melanin is oxidized, it develops an absorption maximum of 470 nm and an emission maximum of 540 nm, suggesting a link between melanin degradation and lipofuscin formation. Lutein and zeaxanthin, xanthophyll carotenoids that appear to play an important role in protecting against age-related macular degeneration, are also found in the retina and have the ability to interact with light. While not bound by any particular theory of operation, it is believed that all of these molecules, which have the capacity to absorb light, would play a role in laser welding/photocoagulation of these tissues in the NIR absorption band of water. The tuning of the laser to the range 1380-1600 is believed to provide optimal heating of water in the tissues and therefore of the molecules that are in the water.

The molecular constituents of the cornea will now be described. The cornea is a transparent nonvascular fibrous layer that covers the front surface of the eye. It is composed of five histologically distinct layers that include the epithelium, Bowman's membrane, stroma, Descemet's membrane, and endothelium. The stroma of the cornea represents 90% of the tissue. Seventy percent of the dry weight of the cornea is the molecule collagen. Glycosaminoglycans molecules are also present in the cornea. Keratin sulfate is the most abundant, at 65% of the total glycosaminoglycans. The remainder are represented by chondroitin and dermatan sulfate. Again, while not bound by any particular theory of operation, it is believed that the collagen molecules and the water molecule are a consideration in the NIR welding process. NIR lasers in accordance with the present invention are believed to elevate the temperature of collagen molecules by energy transfer from water to collagen. The helical collagen molecules partially denature and re-nature to form a real time, water tight, full thickness bond. In order to achieve the above process, the output power of the laser of the exemplary embodiments is believed sufficient to heat the tissue to about 65° C. to allow the collagen fibers to renature. The method of exposure must also be such so as to prevent thermal damage to the tissue.

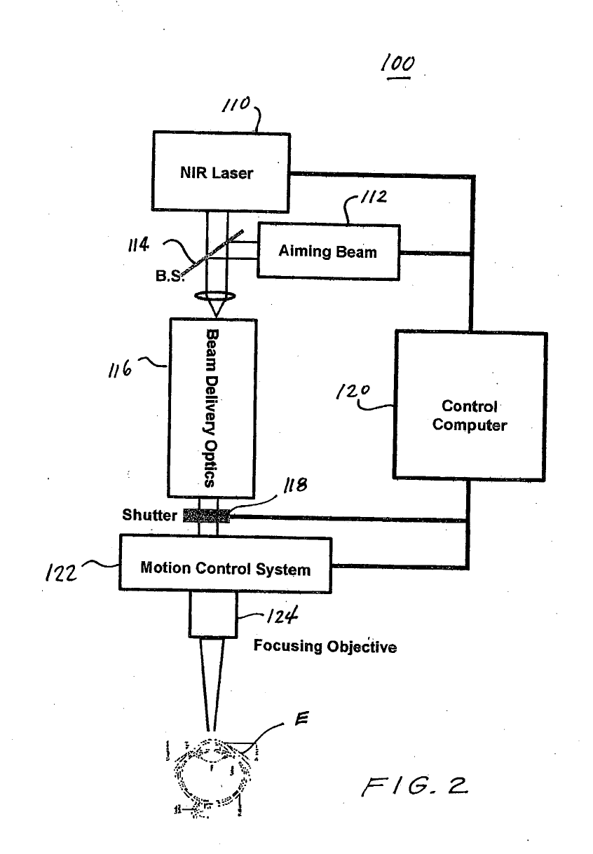

Referring to FIG. 2, a representative laser surgery system 100 in shown. In accordance with the present invention, the NIR laser 110 is any laser which emits a beam in the 1380 to 1600 nm range with the necessary output power, as will be described below. Three lasers which have been used in exemplary embodiments are, e.g., (1) an Erbium fiber laser; (2) a Cr⁴⁺:Ca₂GeO₄ CUNYITE (Calcium Germinate) laser; and (3) a Cr⁴⁺:YAG laser. The erbium fiber laser used in the experiments is a B&WTek model BWF2 emitting at a wavelength of 1455 nm. Erbuim fiber lasers are commercially available from vendors, such as, e.g., B&W Tek, Inc. of Newark, Del. The CUNYITE (Cr⁴⁺:Ca₂GeO₄) laser emits at wavelengths in the range of 1350 to 1500 nm. The Cr⁴⁺:YAG laser emits at wavelengths in the range of 1400 to 1600 nm. Both of these lasers, which are based on Cr⁴⁺ ions, are fully described in U.S. Pat. Nos. 4,987,575 and 4,932,031, which are incorporated by reference in there entirety herein. Other lasers useful for carrying out the invention (e.g., welding tissue) include, for example, semiconductor lasers (InGaAs) operating at 1450-1460 nm. It is understood that this list is not all-inclusive but only presents some of the possible laser sources for welding of ocular tissue in the NIR.

A low power visible light source, e.g., an aiming beam, is coupled into the fiber, and may be a laser, LED or lamp. This visible light beam is used to allow alignment of the NIR light. An exemplary aiming beam 112 for the Erbium fiber laser may be a red LED, which is included as a component in the B&W Tek Laser. The aiming beam for the CUNYITE and Cr⁴⁺:YAG lasers may be a commercially available Helium Neon laser. For the B&W Tek Laser, the beam splitter 114 is integrated into the laser assembly. For the CUNYITE and Cr⁴⁺:YAG lasers, the beam splitter 114 is commercially available from optical component vendors (e.g., Oriel, Melles Griot, Newport Corp.), and its use is well known in the art.

The output of the laser may be coupled into a focusing lens or lens system such as a microscope objective. In the exemplary embodiment, the beam delivery optics 116 for the B&W Tek Laser may include a B&W Tek Optical Fiber, a commercial microscope objective, a light guard, free space optics, and/or other optical components as is well known in the art. The beam delivery optics 116 for the CUNYITE and Cr⁴⁺:YAG lasers may include a focusing lens for coupling the laser light into the optical fiber which delivers the light to the tissue. The focusing lens is a commercial microscope objective. The laser beam may be coupled into a 4-meter long multi mode fiber.

A shutter, e.g., mechanical or electronic, may be used to independently control exposure times of the NIR and visible beams. The shutter 118 of the B&W Tek Laser is integrated into the laser. Shutter control is performed by externally applying a voltage to an input connection on the B&W Tek Laser. Timing is controlled by software which controls the welding beam position, as will be described in greater detail below. The CUNYITE and Cr:YAG lasers in the exemplary embodiment are shuttered by a UniBlitz Model T132 shutter driver controller manufactured by Vincent Associates of Rochester, N.Y.

The fiber (e.g., laser) and focusing lenses may be mounted on a computer controlled precision translation stages, which allow motion along three axes (x, y, z). This translation system controls the position of the laser spot relative to the ocular tissue E. A control computer 120 in the exemplary embodiment is a Dell Optiplex computer, but it is understood that any personal computer running a Windows OS may be used. The motion control software is written in C++. The program was compiled using Microsoft Visual C++ Version 6.0 and the Aerotech SDK. The software program controls the motion of the translation stages, which in turn, control the location and length of the welded incision. The term scanning of the incision of the incision refers to relative movement of the laser and the tissue being welded. The area of the reapproximated tissue being scanned is referred to as the excitation area. The program controls the exposure time and hence the total fluence during welding. The program also controls a laser shutter. The shutter has two functions: (1) it blocks laser exposure after completion of the weld cycle, and (2) it blocks laser illumination for short times during the welding cycle, allowing the tissue to cool. The second shutter function causes “periodic blocking” of the laser beam. The program also controls the spatial pattern made by the welding laser. By moving the translational stage, the laser beam can be trained and moved directly on the incision, or the region in which two sections of tissue are to be joined. Alternatively, the beam may be spatially offset to either side, or both sides, of the incision, or the region in which two sections of tissue are to be joined. The offset pattern is designed to prevent buckling of the tissue by distributing the laser beam and therefore, the heat produced, over a wider area of tissue than that of the laser spot size. The duration of the laser exposure, the duration of cooling cycles, and the spatial pattern of the weld are modified to provide successful welding.

The motion control system 122 of the exemplary embodiment, is manufactured by Aerotech, Inc. of Pittsburgh, Pa., may consist of the following components: Unidex 500 PCI-based controller, DR500 motor driver and chassis, ATS100-050 linear stepping motor, ATS50-50 linear stepping motor, test software and software development kit, cables and brackets. The function of the motion control system is to physically move the tissue under the laser beam in a pattern as directed by the movement software. The system described herein is also useful for treating ocular tissue of live patients. According to this application, modifications are made in the system that are well-known in the art to move at least one or all of the laser 110, the beam delivery optics 116 and/or the motion control system 122 with respect to a stationary patient. The laser system described herein may be used with optical systems already available and currently in use.

The focusing objective 124 in the exemplary embodiment is a commercially available microscope objective, and well-known in the art to direct the beam of electromagnetic radiation onto the patient's eye E. A Thor Labs MeterMate power meter may be used to measure the laser power prior to performing measurements.

Depending on the type of ocular tissue to be treated, two types of welding procedures may be used. A first type of weld may be used when repairing incisions or torn tissue. This welding may be performed by apposing the cut edges of the tissue and focusing the laser beam at the midpoint of the tissue juncture. The laser beam is then moved along the incision, i.e., “scanned,” for example, a 5 mm incision line in a cornea would receive five passes of NIR light over a time period of 55 seconds. A second type of weld is a spot weld. For example, when welding a retina, the light beam is not moved. In this instance, the beam is again focused mid-tissue and a certain fluence of light delivered to a spot in the tissue.

System 100 supplies power sufficient to heat the ocular tissues E to approximately 65 degrees. The laser power and exposure times were adjusted to optimize the strength of the weld. A study on ex vivo porcine eyes was performed to determine the threshold fluence level for tissue damage. In subsequent measurements, the laser power and exposure time were limited to less than the determined threshold in order to prevent tissue damage. (The fluence is defined as the laser power multiplied by the exposure time and divided by the exposure area.) Standard histological analysis of the samples were done using hematoxyn-cosin staining of the ocular tissues. Tissue disruption by charring, vapor vacularization, or molecular denaturing were analyzed using these methods. An optimal weld was considered one that had a full thickness weld that could not be discerned from other normal tissue in the area using these histological techniques. During initial experiments a thermocouple, was placed in the tissue parallel to the laser weld to measure the temperature of the tissues.

### EXAMPLES

The welding system has been used on numerous ocular tissues that include sclera/choroid, retina, and cornea. The following examples illustrate certain properties and advantages inherent in some particular embodiments of the invention.

### Example 1

### Porcine Skin Tissue

Although the apparatus and methods in accordance with the invention are primarily intended for use with treatment of ocular tissues, experiments were also performed on skin tissue. Porcine skin samples were supplied on an ice pack, and subsequently washed and cleaned with 0.9% saline solution. The skin samples were opened by incision along the long axis and rectangular pieces, 20×100 mm dimension, were prepared with the long side of the rectangle along the long axis. The skin samples were also dissected into 20×5 mm pieces. The thickness of the skin samples ranged from 1.45-2.50 mm. The samples were further bisected along the 5 mm side, and the cut edges of two smaller segments were then apposed on a glass slide and placed on the translation stage. Tissue holders connected to the translation stage and equipped with micrometer screws were used to keep the cut edges in apposition during the welding process.

The laser beam was focused mid thickness in the sample along the line of apposition of the two pieces. The Erbium fiber laser was used for this weld. The direction of the weld was parallel to the cut and re-approximated samples. Since the orientation of the fibers in the skin samples was not evident, no relationship between the direction of the weld and the direction of the fibers could be established before the welding procedure. The wavelength of the pulse used in the experiment was 1455 nm. The power of the pulse was about 280 mW, measured with a power meter from Thorlabs Inc. (Model: D10MM). The total laser exposure time of the continuous wave laser was 900 seconds. The spot size was 80 μm in diameter. The light was directed onto the epidermal surface of the tissue and the beam focused at the mid point of the thickness of the tissue, 0.725-1.25 mm.

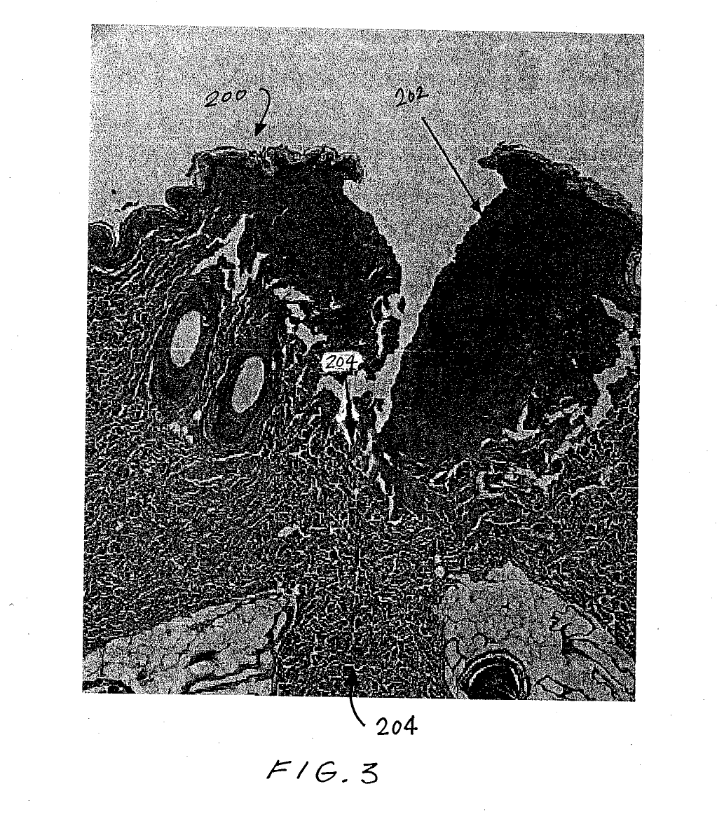

In FIG. 3, the results of this procedure on porcine tissue 200 are shown. Arrow 202 indicates an area of porcine tissue that has been photocoagulated. Arrows 204 indicates a region where the skin tissue has been welded without tissue denaturation. For example, after the welding of lower portion of this tissue, the cut portion of the approximated and welded tissues cannot be identified from the tissues that were not cut when using histology as the end point measurement. On the other hand, photocoagulation implies that thermal effects of the laser have caused destructive irreversible denaturation of molecules within the tissue and that these effects can be seen when using histology. In the Figure, photocoagulation that has taken place during the welding of pigskin tissue in area 202, while welding of the tissue without photocoagulation has taken place in the area between arrows 204.

### Example 2

### Ex Vivo Retina Tissue Treatment

A complete circular incision was made 2 mm lateral to the limbus of the eye. The circular piece of tissue was gently lifted from the eye while using a pair of scissors to cut the collagen fibers vitreous. Care was taken to avoid pulling the retina off of the choroid and sclera. The remaining eye cup was cut into separate 5×5 mm squares. The squares were kept in a moist chamber prior to use.

The continuous wave Erbium fiber laser was used for this welding. The wavelength of the laser used in the experiment was 1455 nm. The power of the laser was 200 mW. During welding, the exposure time was 20 seconds. The laser beam was not moved with respect to the retinal tissue. The spot size was 80 μm in diameter. The light was focused directly onto the lumen surface of the prepared eye cup tissue.

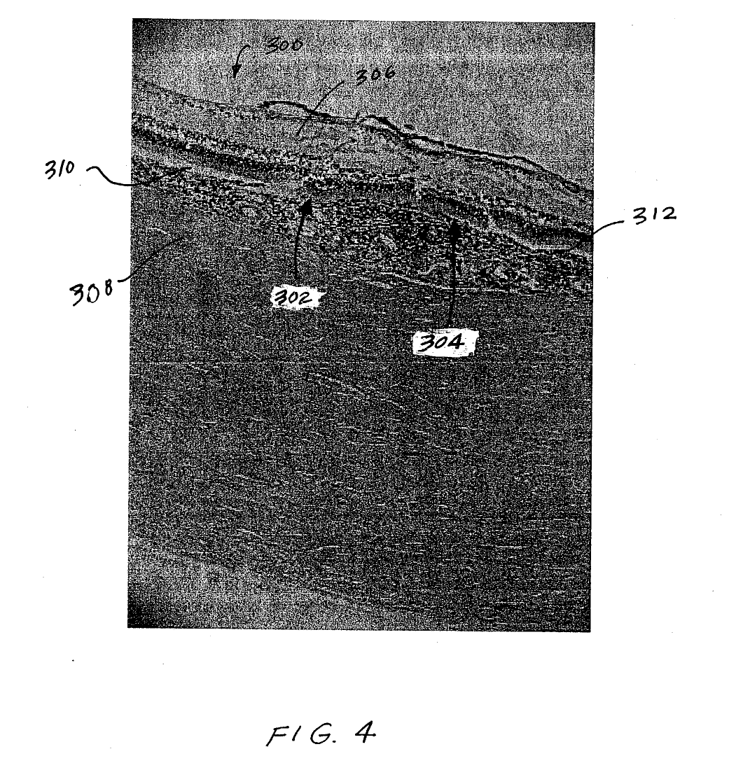

FIG. 4 represents the welding of porcine eye retina to the RPE layer. The central portion of the figure, indicated by arrows 302 and 304, shows retinal tissue 306 attached to the underlying RPE layer 308. To either side of this weld the retinal tissue remains unattached to the RPE layer (regions marked 310 and 312).

### Example 3

### Ex Vivo Corneal Tissue Treatment

A complete circular incision is made 2 mm lateral to the limbus of the eye. The circular piece of ocular tissue is gently lifted from the eye while using a pair of scissors to cut the collagen fibers in the vitreous. The lens, iris, and aqueous humor are removed from the circular piece of ocular tissue. A piece of corneal tissue (preferably the largest piece possible) is cut in the form of a rectangle from the remaining piece of circular tissue that includes a circular piece of the sclera and the attached cornea. The remaining rectangular piece of tissue of cornea is then bisected down the long axis of the tissue. These two rectangular pieces of cornea are then bisected parallel to the short edge of each of the tissues. Each pair of bisected tissues is then re-approximated and welded.

The Erbium fiber laser was used in this experiment. The wavelength of the pulse is 1455 nm. The power of the pulse applied is 150 mA. The point of laser light was delivered along the edges of the cut surfaces. The tissue was moved back and forth under the NIR light with a translation stage. Five passes of light were delivered over the approximated incision for a total duration of 115 seconds. The light used in this example is a continuous beam, not pulsed, and the tissue moves under the continuous beam. The spot size was 80 mm in diameter. The light path was directed onto the exterior surface of the cornea to a midpoint depth of 1 mm (one half the total depth of 2 mm). Allowing for the percent water content of 80%, the depth of penetration is calculated to be 0.400 mm.

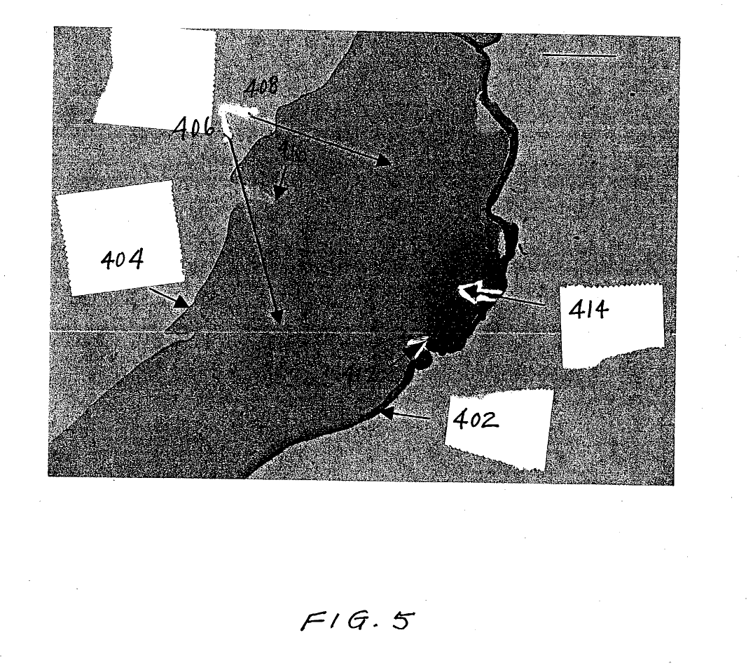

FIG. 5 illustrates the NIR light welding of the porcine cornea. The epithelium 402 and endothelium 404 are pictured. A beam of laser NIR light was directed onto the cut edges of two pieces of cornea tissue 406 and 408 that were reapproximated after they were bisected using a microtome blade. Five passes of light were delivered over the reapproximated edges of the tissue. As illustrated in the figure, there is a full-length seamless weld of the stromal tissue in the area indicated by arrows 410 and 412. At this power and time of treatment of the tissue there was some denaturation of the stroma, e.g., region 414.

### Example 4

### Ex Vivo Scleral/Choroidal Tissue Treatment

As in Example 3, above, complete circular incision was made 2 mm lateral to the limbus of the eye. The circular piece of tissue was gently lifted from the eye while using a pair of scissors to cut the collagen fibers vitreous. The retina was carefully peeled off of the choroid and sclera. The remaining eye cup was cut into separate 5×5 mm squares. The squares were kept in a moist chamber prior to use.

The continuous wave Chromium:YAG laser was used for this welding. A point weld was done using a wavelength of 1465 nm at a power of 400 mW for 20 seconds.

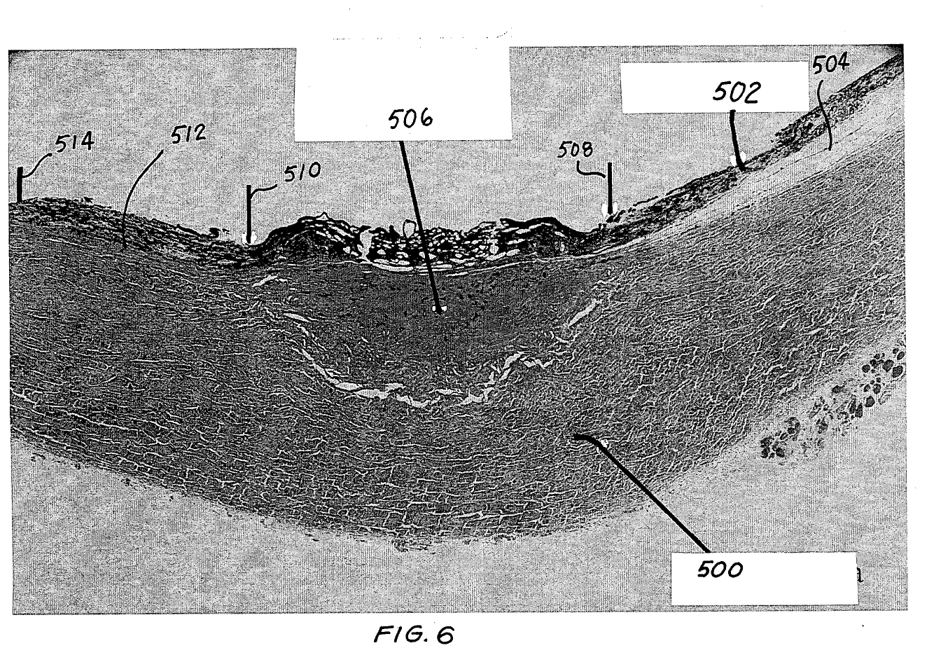

FIG. 6 illustrates a welding and photocoagulation of the scleral tissue 500 and the choroidal tissue 502. A region of interlayer splits between the choroidal tissue 502 and the scleral tissue 500 are indicated by arrow 504. The area 506 (between arrows 508 and 510) has been adhered by photocoagulation of the two tissues. The area 512 (between arrows 510 and 514) has been welded.

### Example 5

### Ex Vivo Scleral Tissue Treatment

The Scleral tissue was prepared as described for the Scleral/Choroid tissue of Example 4. However, in this experiment the tissue was cut into 5×10 mm rectangles. An incision was made down the center of the tissue parallel to the long axis. Two mm sections were left uncut at each long end of the tissue to insure good approximation of the cut edges of the sclera and choroid. The tissue was placed on a glass side and moved under the Erbium laser beam.

The 9.3 mm incision in the sclera was welded together using the Erbium laser at a wavelength of 1455 nm at a power of 110 mW for a total exposure time of 110 seconds. During this time period there were ten passes of the laser over the incision area.

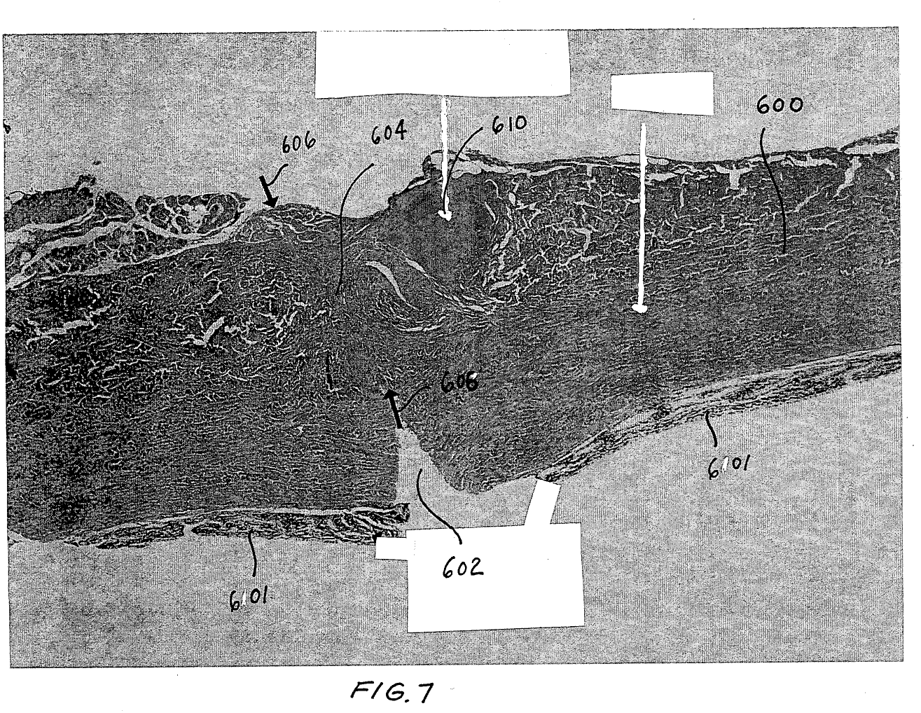

FIG. 7 illustrates a section of scleral tissue 600 and choroidal tissue 601. An incision though the tissue was repaired with a weld. The unwelded portion of the incision is the area indicated by arrow 602. The area of the weld 604 lies between arrows 606 and 608. An area of photocoagulation 610 is also shown.

It will be understood that the foregoing is only illustrative of the principles of the invention, and that various modifications can be made by those skilled in the art without departing from the scope and spirit of the invention.

What is claimed is:

1. A method for welding of ocular tissue comprising:
exposing ocular tissue to a beam of electromagnetic radiation tuned to an NIR wavelength between about 1380 nm and about 1600 nm in the water absorption band.

2. The method of claim 1, wherein the step of exposing ocular tissue to a beam of electromagnetic radiation comprises providing a laser.

3. The method of claim 1, wherein the step of exposing ocular tissue comprises exposing retina tissue to said electromagnetic radiation.

4. The method of claim 1, wherein the step of exposing ocular tissue comprises exposing cornea tissue to said electromagnetic radiation.

5. The method of claim 1, wherein the step of exposing ocular tissue comprises exposing a choroid layer and scleral tissue having an interlayer split therebetween to said electromagnetic radiation.

6. The method of claim 1, wherein the step of exposing ocular tissue comprises exposing scleral tissue to said electromagnetic radiation.

7. The method of claim 1, wherein the step of exposing ocular tissue comprises scanning an excitation area of said ocular tissue with said electromagnetic radiation; and periodically blocking the excitation area from the electromagnetic radiation.

8. The method of claim 7 further comprising exposing said ocular tissue to the electromagnetic radiation in a region in which sections of ocular tissue are to be joined.

9. The method of claim 7 further comprising exposing said ocular tissue to the electromagnetic radiation in a region that is spatially offset from the region in which sections of ocular tissue are to be joined.

10. An apparatus for welding of ocular tissue comprising:
a source of electromagnetic radiation configured to deliver a beam to the ocular tissue which is tuned to an NIR wavelength between about 1380 nm and about 1600 nm in the water absorption band.

11. The apparatus of claim 10, wherein the source of electromagnetic radiation comprises a laser.

12. The apparatus of claim 11, wherein the source of electromagnetic radiation comprises an erbium fiber laser.

13. The apparatus of claim 11, wherein the source of electromagnetic radiation comprises a laser incorporating a Cr⁴⁺: doped crystals.

14. The apparatus of claim 10, further comprising:
a motion control system for moving said source of electromagnetic radiation relative to an excitation area of said ocular tissue.

15. The apparatus of claim 10, further comprising:
a shutter configured to periodically block the excitation area from the electromagnetic radiation.

16. The apparatus of claim 10, wherein the beam delivered by the source of electromagnetic radiation is delivered to the region in which sections of ocular tissue are to be joined.

17. The apparatus of claim 10, wherein the beam delivered by the source of electromagnetic radiation is delivered to a region that is spatially offset from the region in which sections of ocular tissue are to be joined.

18. The apparatus of claim 10, wherein the ocular tissue comprises retina tissue.

19. The apparatus of claim 10, wherein the ocular tissue comprises cornea tissue.

20. The apparatus of claim 10, wherein the ocular tissue comprises scleral/choroid tissues.

21. The apparatus of claim 10, wherein the ocular tissue comprises scleral tissue.
